(19) United States
(12) Patent Application Publication
(10) Pub. No.: US 2008/0188790 A1
(43) Pub. Date: Aug. 7, 2008

(54) Apparatus and Method for Dosage of Additives While Collecting a Liquid

(76) Inventors: Per Olsson, Stockholm (SE); Erik Ohlin, Stocksund (SE); Kjell Radegran, Solna (SE)

Correspondence Address: STITES & HARBISON PLLC, 1199 NORTH FAIRFAX STREET, SUITE 900, ALEXANDRIA, VA, 22314, US

(21) Appl. No.: 11/816,066

(22) PCT Filed: Feb. 16, 2006
(86) PCT No.: PCT/SE06/00206

§ 371 Date: Aug. 10, 2007

(30) Foreign Application Priority Data
Feb. 16, 2005 (SE) 0500363-7

(51) Int. Cl. A61M 1/02; A61M 1/00; G05D 7/00

(52) U.S. Cl. 604/4.01; 435/2; 137/98

(57) ABSTRACT

The invention relates to an apparatus for sucking liquid, in particular blood, from a collecting location, through a suction conduit (4) having an inlet end (5), wherein a liquid additive, in particular a CPD solution, is fed through an outlet end (15) of a conduit for additives (13). The inlet end (5) and the outlet end (15) being fixated in respect of and close to each other such that the additive is fed out to the collection location (16) close to the inlet end (5) and is mixed with the liquid that is being sucked before it enters the inlet end. A dosage unit (12) is controlled in dependence of the sub pressure in the suction conduit (4) for to regulate the feeding of the additive in dependence of the quantity of liquid being sucked.

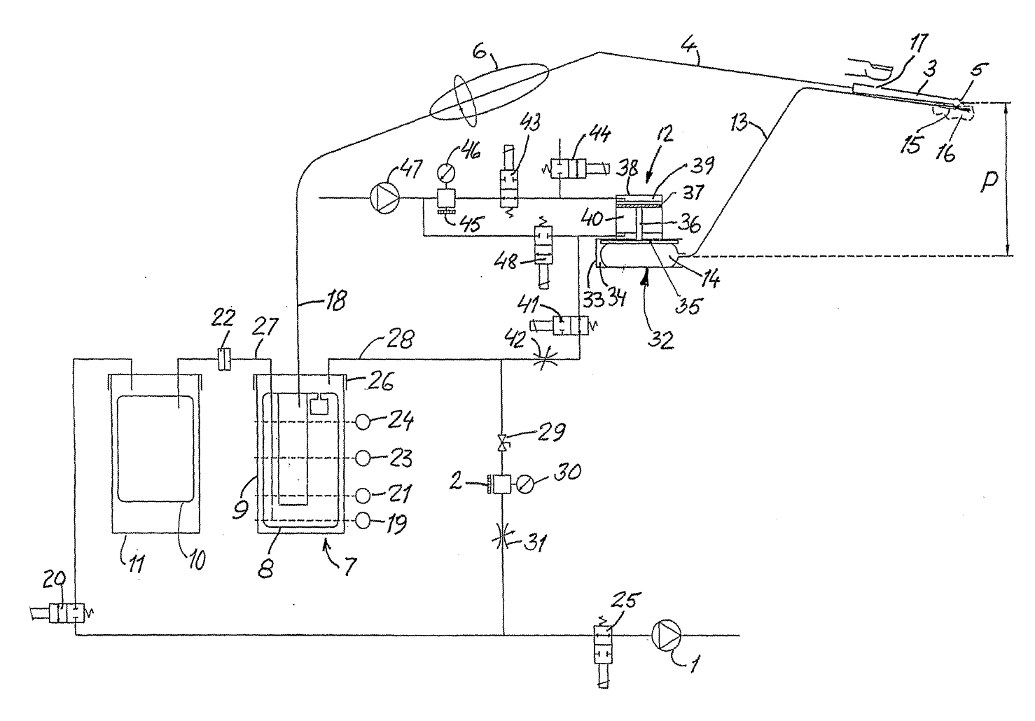

### FIELD OF THE INVENTION

The present invention relates to an apparatus and a method for dosage of additives while collecting liquids of different kinds for storage in one or several closed packages, such as transfer bags, or for transmittal to another fluidal system. The invention is especially suitable for treatment of blood during surgical operations but may also be used in treatments of other liquids prone to deterioration, oxidation or affection of any kind from the presence of air, gas or similar or during treatment of liquids that in them selves are harmful. Examples of such liquids are victuals, liquids that are caustic or hazardous to the environment, oils, waste liquors and others.

The invention is especially suitable for treatment of blood and therefore it will in the following mainly be described in connection with such treatment.

### BACKGROUND

Blood for use at transfusions of blood is constantly an article in short supply. Large quantities of blood are used up during transfusions of blood, for instance in relation to surgical operations. Collecting, testing and storage of transfusional blood is costly. The blood the patient loses during a surgical operation is normally not collected. This lost blood must be replaced by transfusions and it is not excluded that several litres of blood has to be replaced. Additionally, there exists a risk of transmission of infections due to transfusions of blood.

In order to reduce the problem with shortage of transfusional blood one might, during surgical operations, collect as much as possible of the patients own blood that is being lost from the circulatory system during the operation and recirculate it to the patient. The blood must in such cases be treated without risk for contamination throughout the treatment.

Autologous apparatuses for collecting liquids are disclosed in i.e. SE 467 725, EP 0 742 737 and SE 515604. In all these apparatuses the blood lost during the surgical operations is sucked up continuously with a suction conduit. A coagulation restricting additive is added automatically and in proportion to the amount of blood that has been collected in a specific moment. The blood is then allowed to pass through a closed system via suitable filters for defoaming and separation and recipients for separation of bubbles enclosed in the blood and for filtration. The blood is then collected in containers, e.g. blood bags, with no intervention what so ever of air or other gases, using a sub pressure in the apparatus for collecting the liquid. A suitable sub pressure in the apparatus for collecting liquid is created by connecting it to a source of sub pressure in form of a suction pump.

The dosage of anticoagulant is performed before the blood arrives to the means for filtration and deaerating. In conventional apparatuses for collecting liquids the dosage of anticoagulant is done during the suction in proportion to the quantity of sucked blood using dosage devices controlled by the sub pressure in the suction conduit.

In these apparatuses anticoagulant is added to the blood after transportation from a suction end or inlet end on the suction conduit to a point in the suction conduit where an outlet end of a conduit for supply of anticoagulant is connected. The section of the suction conduit located between the inlet end and this point must therefore be coated with a material that is compatible to blood, e.g. heparin, such that the blood does not coagulate or is harmed in any other way before it reaches the point where the anticoagulant is supplied. The provision of a coating of a material that is compatible to blood, i.e. a heparin treatment, is complicated to achieve, and parts treated in this manner are therefore expensive. Additionally, it is it difficult to control that the heparin treatment was successful, hence there is a risk that the blood is infected due to an unsuccessful heparin treatment. As the suction conduit is a disposable product which is thrown away after having been used only once, every measure eliminating the need of a heparin treatment implies a significant reduction of costs.

Tests have been performed using a separate nozzle to apply anticoagulant to a gaping wound from which blood is sucked for allowing the blood to mix with the anticoagulant before it is sucked into the suction conduit. With an effective method of this kind the need of treating the suction conduit with heparin would be eliminated. It is however difficult to achieve the rather high accuracy that is required for the dosage of the anticoagulant. The proportion of anticoagulant in the blood should be held inside the interval of 5-15%, and should preferably be between 8-12%. Up to this day it has been impossible to achieve such an accuracy. Firstly, it is difficult to manually adapt the supply of anticoagulant to the quantity of blood being sucked at a specific moment. Additionally, it is difficult to add the anticoagulant exactly where the blood is being sucked throughout the whole suction operation, such that the blood in deed is thoroughly mixed with the anticoagulant before it enters the suction conduit.

### SHORT DESCRIPTION OF THE INVENTION

The object of the invention is to solve the problems described above and provide an automatic and accurate dosage of additives to a sucked liquid, in particular blood, in such a way that the additive is reliably supplied to the liquid before it enters the conduit, together with the liquid that is being sucked, such that no treatment is required for any part of the apparatus, i.e. a heparin treatment in the case the liquid to be collected is transfusional blood.

This object is achieved with an apparatus according to claim 1 and a method according to claim 6. The independent claims provides favourable embodiments of the apparatus and the method according the invention.

According the invention the collecting of the liquid and the supply of the additive to the liquid is performed in conduits, one suction conduit and one conduit for the supply of additives, which are connected in such a way that the inlet end of the suction conduit and the outlet end of the supply conduit for additives are placed in such a manner that the latter is close to the place where the liquid is collected throughout the whole sucking operation. Both of the conduit ends may at all times be handled as a unit, ensuring that the additive is fed to the liquid at the collecting location and that the liquid and the additive are being sucked collectively, in other words the collected liquid contains the additive already when it enters the inlet end of the suction conduit.

As for conventional apparatuses the supply of additives is controlled as a function of the sub pressure achieving the sucking of the liquid through the suction conduit. It is however not possible to use the same arrangement as for the conventional apparatuses, as the outlet of the conduit for additives in these apparatuses are affected by a surrounding sub pressure throughout the sucking, whereas in the apparatus and the method according to the invention the outlet end of the conduit for additives is placed outside the system at atmospheric pressure. In conventional apparatuses additives are sucked out from the outlet of the conduit for additives as soon as the sub pressure increases in the suction conduit, wherein the quantity of additives is proportional to the sub pressure. In the apparatus according the invention the flow dosage of the additive is also controlled in dependence of the sub pressure, but the sub pressure is converted into a overpressure achieving the transport of additives in the conduit for additives, whereby the additive is forced out towards the liquid at the collecting location as soon as the sub pressure increases in the suction conduit.

In a preferred embodiment of the invention the sub pressure is adapted to control a pressure element, applying an increasing pressure on a storage of additives as a result of an increasing sub pressure in the suction conduit to which the conduit for additives is connected, such that the storage of additives is exposed to an increasing pressure as a result of an increasing sub pressure in the suction conduit, whereby an increased flow of collected liquid results in a proportional increase of added additive.

### DETAILED DESCRIPTION OF THE PREFERRED EMBODIMENT

In the following an example of an embodiment of the invention will be described with reference to the enclosed figure, which schematically shows an apparatus according the invention adapted for collecting blood parting from the circulatory system of a patient during a surgical operation.

The invention particularly relates to the dosage of additives, in particular an anticoagulant, i.e. a solution containing citrate-phosphate-dextrose, CPD solution, to the liquid to be collected; the liquid that is collected consists in this case of blood with added CPD solution. For better understanding of the invention a description of the general structure and function of the apparatus as a whole will open the detailed description.

The apparatus for collecting liquid in the figure operates with a certain predetermined pressure gradient for providing a sub pressure (suction) in the apparatus. The sub pressure is created by a suction pump 1 with a capacity of sucking up to e.g. 3500 ml blood per min and is preferably controllable by a sub pressure regulator 2, such that the apparatus is operable at different pressure levels. The pump is connected to the different parts of the apparatus by means of a tubing or hose system with valves, manometers and pressure transducers of conventional types with known features and functions and are therefore not described closer herein.

The apparatus includes the following main parts: a holding device 3 with a flexible suction conduit 4 in form of a hose with a free, open inlet end 5 for collecting blood, a mixing and defoaming unit 6 for mixing the collected blood with the CPD solution and separating the foam, a recipient 7 with a recipient bag 8 placed inside a rigid container 9, a transfer bag 10 placed inside a rigid container 11, and a dosage unit 12 including a flexible conduit (a hose) for additives 13 connected or connectable to a storage of additives in the form of a flexible bag, i.e. a CPD bag 14, having an open outlet end 15 through which the additive in form of CPD solution may be transferred to the blood at the moment it is being sucked up through the suction conduit 4 and conveyed to the recipient bag 8 and the transfer bag 10. The holding device 3 keeps the inlet end 5 of the suction conduit 4 and the outlet end 15 of the conduit for additives 13 fixed in a predetermined position close to each other.

At least the parts 4, 5, 6, 8, 10, 13, 14 and 15 of the shown exemplary embodiment, which corresponds to the parts that contacts the blood and the anticoagulant, are presented as a disposable assembly, i.e. adapted to be used only once. The holding device 3 may a separate part, but it may also be a part of the disposable assembly.

The blood is sucked from a collection location 16, e.g. a gaping surgical wound, by means of the inlet end 5 of the holding device 3, which is provided with a finger hole 17 to be kept closed in order to be able to suck any liquid through the suction conduit 4, and passes through the suction conduit 4 to the mixing and defoaming unit 6. This unit 6 includes a coarser suction conduit section 18, which is rotated in a known manner in order to carefully mix the collected blood and the added CPD solution in the suction conduit and separate any air bubbles from the blood. The coarser dimension of the section 18 of the suction conduit is withheld all the way down to the recipient bag 8.

The mixed blood from the unit 6 is sucked down into the 2000 ml capacity recipient bag 8. A sensor 19 gives a signal when the quantity of collected blood reaches a minimum level at about 200 ml. Only at this level is it safe to transfer blood to the transfer bag 10 without risking that air is transferred with the blood. The transfer operation is launched by the opening of a magnetic valve 20. It is however not suitable to transfer blood until a second sensor 21 has given a signal indicating that there is 900 ml in the recipient bag. Only at this level is it namely possible to fill up a whole transfer bag 10, which normally has a capacity of 700 ml, without risking that the blood level goes below the above mentioned minimum level at 200 ml. Once the transfer bag 10 is filled the magnetic valve 20 is shut, wherein the container 11 (via an air inlet on the magnetic valve 20) is connected to atmospheric pressure, whereby air is allowed to enter in the space surrounding the transfer bag 10 and whereby the sub pressure around it is eliminated. Thereby, the removal of the lid of the container 11 is facilitated, as the lid otherwise would be retained by the sub pressure in the container 11. The atmospheric pressure outside the bag 10 is also helpful for pressing out any air bubbles form at the top of the bag 10. Next, a hose valve 22 is shut, wherein the recipient bag 8 is once again filled. The suction system may hence be used without interruption during the exchange of transfer bags 10 as the recipient bag 8 has a spare capacity of at least 1300 ml. The transfer bag 10 may of course be exchanged several times why the capacity of the apparatuses in principle is unlimited. In some applications it may be suitable to only fill the recipient bag 8. Another sensor 23 in the recipient 7 indicates when the content in the recipient bag 8 exceeds 1600 ml, wherein two transfer bags (the shown transfer bag 10 and another not shown transfer bag, connected after the first) may be filled. Yet another sensor 24 indicates when the content reaches approximately 2000 ml and the recipient bag 10 is almost full. If this level is exceeded the magnetic valve 25 is shut, wherein the atmospheric pressure is attained in the system and no more blood may be collected.

The container 9 is provided with a lid 26, which has openings for a suction conduit section 18 arriving from the mixing and defoaming unit 6, an outlet conduit 27 for removal of liquid, and a sub pressure conduit 28. The suction conduit section 18 debouches into the upper part of the recipient bag 8, while the outlet conduit 27 reaches down into the bottom of the recipient bag. The sub pressure conduit 28 debouches just above the recipient bag 8 and is in fluid communication with the suction conduit 4 via an integrated filter.

The sub pressure conduit 28 is connected to the suction pump 1 via a closing valve 29, a manometer 30 and the sub pressure regulator 2. Hence the container 9 and consequently also the suction conduit 4 is affected by the sub pressure in the suction inlet of the sub pressure conduit 27. The sub pressure is controllable by means of the sub pressure regulator 2. Further the sub pressure conduit 28 contains a needle valve 31.

The dosage unit 12 includes a holding fixture or box 32 with rigid housing 33 and a space 34 with room for a CPD bag 14 from which CPD solution shall be transferred to and out through the outlet end 15 of the conduit for additives 13. The holding fixture or box 32 is preferably placed such that it is easily accessed for replacement of CPD bags 14.

Further, the dosage unit 12 has a pressure element 35 arranged to apply a variable pressure towards the CPD bag for pressing an appropriate quantity of CPD solution through the conduit for additives 13 and the outlet end 15 when, and only when, blood is being sucked from the collecting location 16 and through the inlet end 5 of the suction conduit 4. The pressure element 35 is connected via a rod 36 to a movable wall in the form of a piston plate 37. The piston plate 37 is slidably arranged inside a cylindrical regulating device 38 and divides it in an upper chamber 39 and a lower chamber 40.

The lower chamber 40 is connected to the suction pump 1 via a magnetic valve 41 and a needle valve 42, whereby a sub pressure reigns in the suction conduit 4 that is a proportional to the sub pressure in the lower chamber 40. The upper chamber 39 is connected to a pressure source 47 (which may be the pressure part of the suction pump 1) via two magnetic valves 43 and 44, a pressure regulator 45 and a manometer 46 and is pressurised to a higher pressure than the pressure reigning in the lower chamber 40. Also, the pressure pump 47 is connected to the lower chamber 40 via a magnetic valve 48. This valve is however normally closed and is only opened when the CPD bag needs to be accessed, e.g. when it is empty end needs to be replaced. In order to make it possible to allow two different pressures reign in the different chambers 39 and 40 the piston plate 37 is arranged tight against the inside of the regulating device 38.

As mentioned above it is important that the anticoagulant, the CPD solution, is dosed continuously in an appropriate proportion to the quantity collected blood. This dosage is achieved by the dosage unit 12.

When the apparatus for collecting liquid is sucking air or gas alone the additive in the conduit for additives 13 is arranged to just reach the outlet end 15 provided that it is held at the height in relation to the dosage unit 12 for which it has been calibrated. The pressure applied by the pressure element 35 on the CPD bag 14 shall hence be adjusted such that no CPD solution is fed through the outlet end 15 of the conduit for additives. The pressure on the CPD bag 14 must therefore differ from the atmospheric pressure at the outlet end 15 with a quantity that corresponds to the pressure difference relating to the height difference between the CPD bag and the outlet end 15 when placed on the height of the collecting location 16. This pressure difference is represented by the arrow P in the figure. The adjustment is made in the following manner.

When the apparatus for collecting liquid is to be used the first action taken is to start the suction pump 1. The closing valve 29 shall be held closed at this moment. The magnetic valve 25 is, as in general use, in the shown open position. A sub pressure is generated all the way to the magnetic valve 20, positioned in the shown general use closed position, and to the pressure regulator 2. Using the pressure regulator the sub pressure is adjusted to a appropriate value, i.e. about 150 mbar. Normally, the needle valve 31 does not need to be adjusted. Thereafter the closing valve 29 is opened such that the sub pressure is spread to the sub pressure conduit 28, the container 9, suction conduit section 18, the mixer 6 and the rest of the suction conduit 4, wherein air is sucked in through the inlet end 5. At the same time a sub pressure is spread to the lower chamber 40 of the regulating device 38 via the needle valve 42 and the magnetic valve 41. The magnetic valve 48 is closed.

When the sub pressure has been created it is time to calibrate the dosage unit. This is done by starting the pressure pump 47 and by adjusting the pressure regulator 45 such that an appropriate pressure is achieved in the upper chamber 39. The magnetic valve 43 is open while the magnetic valve 44 is closed. Due to the sub pressure in the lower chamber 40 a certain pressure is already exerted towards the CPD bag 14. A suitable pressure on the CPD bag 14 may be adjusted by regulating the pressure in the upper cylinder chamber 39 by means of the pressure regulator 45, such that no CPD solution leaves the outlet end 15 even though the conduit for additives 13 is filled all the way up to the outlet end. The pressure in the upper cylinder chamber 39 shall be adjusted such that CPD solution is emitted from the outlet end 15 of the conduit for additives as soon as the pressure decreases in the lower chamber, which it does when liquid is being sucked into the suction conduit 4. It goes without saying that the outlet end 15 of the conduit for additives 13 must be located at the height of the predetermined collecting location 16 during adjustment.

For as long as the apparatus for collecting liquid is in use it is of course important that the outlet end 15 of the conduit for additives 13 is filled all the way to the end even when no blood is collected, such that CPD solution is fed instantaneously when the sub pressure in the suction conduit 4 increases due to the suction of liquid into the inlet end 5 of the suction conduit. As an alternative the calibration may be performed such that CPD solution is fed as soon as the finger hole 17 is shut and that this is to be done just before the inlet end of the suction conduit contact the blood.

Due to the pressure pump 47 and the pressure regulator 45 the pressure in the upper chamber 39 is always kept constant even though the volume of this chamber is continuously increased as the CPD bag 14 is being emptied. The pressure in the lower chamber 40 is likewise kept constant by means of the suction pump 1 and the sub pressure regulator 2, except for the pressure drops implied when liquid is being sucked through the suction conduit 4. These pressure drops results in the desired increase of the pressure on the CPD bag 14 and consequently the desired dosage of CPD solution to the blood that is being collected.

An ordinary CPD bag normally lasts for of 2.5 litres of blood. A sensor (not shown) when the quantity of anticoagulant in the bag is below a certain minimum level, i.e. when the bag is almost empty, e.g. when 5% (30-40 ml) of the liquid is left in the bag. The sensor may also be adapted to sense and alert if there is no bag in the container.

When the CPD bag 14 is empty and shall be replaced the magnetic valve 41 connecting the lower chamber 40 to the suction pump 1 is closed. Then, the magnetic valve 43 connecting the upper chamber 39 to the pressure pump 47 is closed, and instead the magnetic valve 44 is opened, whereby the upper chamber 39 is put under the external atmospheric pressure. Eventually the magnetic valve 48, which connects the lower chamber 40 to the pressure pump 47, is opened. When this chamber 40 is put under pressure the piston plate 37 will be pressed upwards collectively with the rod 36 and the pressure element 35. Thus the space below the pressure element 35 is liberated, such that the CPD bag 14 may easily be replaced. When the new CPD bag is at location no new calibration is needed, provided that the collecting location 16 is at the height for which the previous calibration was done.

The invention is not limited to the exemplary embodiment disclosed herein, on the contrary it may be varied within the scope of the following claims. For instance all means for adjusting, regulating and controlling have deliberately and for reasons of understanding been described in as little details as possible. It is of course possible to construct the apparatus for collecting liquid such that it becomes more user friendly by e.g. automatization of the calibration and other regulating operations.

1. Apparatus for suction of a liquid from a collecting location, which apparatus includes:
a suction conduit (4) having an inlet end (5) and being connected to a sub pressure source (1) for creating a sub pressure in the suction conduit (4), and
a dosage unit (12) for continuously during suction dosing additives to the liquid in a quantity that is proportional to the quantity of sucked liquid, which dosage unit includes:
a conduit for additives (13) being connectable to a storage of additives (14) and having an outlet end (15) for feeding the additive to the liquid, and
a regulating device (38) for regulating the flow of additives from the outlet end (15) of the conduit for additives (13) in dependence of the sub pressure in the suction conduit (4), the outlet end (15) of the conduit for additives (13) being arranged close and in a fixed position with respect to the inlet end (5), such that the additive is fed to the liquid at the collecting location (16) simultaneously as the liquid is being sucked, the additive being sucked through the inlet end (5) of the suction conduit (4) together with the liquid,
characterised in that the regulating device (38) includes a pressure element (35) controlled by the sub pressure in the suction conduit, which pressure element applies an increasing overpressure to the additive in the storage (14) of additives, the increase of the overpressure relating to an increase of the sub pressure in the suction conduit (4), for to increase the flow of additives from the outlet end (15) of the conduit for additives (13) in proportion to the increase of the sub pressure in the suction conduit.

2. Apparatus according to claim 1, characterised in that the regulating device (38) has a first closed chamber (40), which is connected to the sub pressure source (1) and the suction conduit (4), and circumscribed by a movable wall (37) connected to the pressure element (35).

3. Apparatus according to claim 2, characterised in that the regulating device (38) includes a load application device (37, 39) for application of an adjustable preload pressure to the additive in the storage of additives (14).

4. Apparatus according to claim 3, characterised in that the load application device (37, 39) has a second closed chamber (39), which is connected to an adjustable overpressure source (47, 45) and parted from the first chamber (40) by the movable wall (37).

5. Method of sucking liquid, in particular blood, from a collecting location and during the sucking continuously adding liquid additives to the liquid, an open inlet end.(5) of a suction conduit (4) being placed at the collecting location (16) and the additive being fed from a storage of additives (14) trough an open outlet end (15) of a conduit for additives (13), which is connected to the storage of additives, the additive being fed to the liquid at the collecting location, such that the liquid and the additive is being sucked collectively through the inlet end (5) of the suction conduit (4), wherein this end and the outlet end (15) of the conduit for additives (13) are kept in a fixed position with regard to each other,
characterised by that the quantity of additives being fed during the sucking is regulated in proportion to the sub pressure in the suction conduit (4) and thus the quantity of sucked liquid, a pressure being applied to the additive in the storage of additives (14), which is proportional to the sub pressure.
